(19) United States
(12) Patent Application Publication
(10) Pub. No.: US 2015/0005799 A1
(43) Pub. Date: Jan. 1, 2015

(54) RENAL NERVE MODULATION BALLOON HAVING IMPROVED ROBUSTNESS

(71) Applicant: BOSTON SCIENTIFIC SCIMED, INC., MAPLE GROVE, MN (US)

(72) Inventors: JEFFREY S. LINDQUIST, MAPLE GROVE, MN (US); CASS A. HANSON, ST. PAUL, MN (US); DEREK C. SUTERMEISTER, HAM LAKE, MN (US); DANIEL T. QUILLIN, EDEN PRAIRIE, MN (US); PETER G. EDELMAN, MAPLE GROVE, MN (US); AJAY GUPTA, LITTLE CANADA, MN (US); JOSEPH A. ZIELINSKI, PLYMOUTH, MN (US); GARY J. PEDERSON, JR., ALBERTVILLE, MN (US); LIZA J. DAVIS, ST. MICHAEL, MN (US); HENRY H. LEE, MISSION VIEJO, CA (US)

(21) Appl. No.: 14/316,352

(22) Filed: Jun. 26, 2014

Related U.S. Application Data
(60) Provisional application No. 61/839,971, filed on Jun. 27, 2013.

(51) Int. Cl. A61B 18/00; A61B 18/18; A61B 18/08; A61B 18/14

(52) U.S. Cl. CPC A61B 18/00; A61B 18/1492; A61B 18/1815; A61B 18/082; A61B 2018/00434; A61B 2018/0022
USPC 606/192; 156/272.8

(57) ABSTRACT

A balloon for renal nerve modulation, the balloon comprising a polymer material forming a balloon wall having an interior surface and an exterior surface and flexible circuits adhesively bonded to the exterior surface of the balloon wall, wherein the exterior surface comprises laser induced microstructures on at least a portion of said exterior surface.

### CROSS-REFERENCE TO RELATED APPLICATIONS

This application claims priority under 35 U.S.C. §119 to U.S. Provisional Application Ser. No. 61/839,971, filed Jun. 27, 2013, the entirety of which is incorporated herein by reference.

### BACKGROUND

The present disclosure is directed to devices for percutaneous renal artery denervation, particularly expandable balloons and to methods of making and using the same.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal artery denervation can reduce blood pressure, and may be a viable treatment option for many patients with hypertension who do not respond to conventional drugs.

Ultrasound, radiofrequency energy, microwave energy, direct heating elements, and balloons with heat or energy sources may be applied to a region of sympathetic nerves.

A specific method for treatment of the renal sympathetic nerves involves a percutaneous, catheter-based therapy that uses radiofrequency energy to disrupt the renal sympathetic nerves. This method involves the use of an expandable medical balloon which is advanced to the treatment site, expanded, and energy is transmitted through the balloon via flexible circuits disposed on the outside of the balloon.

There remains a need in the art for improved balloons for renal artery denervation having high robustness.

### BRIEF SUMMARY

In one aspect, the present disclosure relates to a balloon for renal nerve modulation, the balloon comprising a polymer material forming a balloon wall having an interior surface and an exterior surface and flexible circuits adhesively bonded to the exterior surface of the balloon wall, wherein the exterior surface comprises laser induced microstructures on at least a portion of said exterior surface.

In another aspect, the present disclosure relates to a flexible circuit, the flexible circuit having an inner surface and an outer surface, the inner surface of the flexible circuit is adhesively bonded to at least a portion of an outer surface of a balloon for renal nerve modulation, the flexible circuit comprising laser induced microstructures on the inner surface that is adhesively bonded to at least a portion of the outer surface of the balloon.

In another aspect, the present disclosure relates to a method of making a balloon for renal nerve modulation, the method comprising providing a balloon having a balloon wall, the balloon wall comprising a polymer material, the balloon having a body, waist and cone portions, the balloon having an interior surface and an exterior surface, removing material from the exterior surface of the balloon by laser etching, the laser etching forms laser induced microstructures in the exterior surface of the balloon and adhesively bonding flexible circuits to the exterior surface of the balloon on at least a portion of the balloon comprising the laser induced microstructures.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a side view of a catheter having a renal nerve modulation balloon disposed on the distal end thereof.

FIG. 2 is a radial cross-section taken at section 2-2 in FIG. 1.

FIG. 3 is a radial cross-section taken at section 3-3 in FIG. 1.

FIG. 4 is a side view of an embodiment of a renal nerve modulation balloon having flexible circuits disposed thereon.

FIG. 5 is a side view of another embodiment of a renal nerve modulation balloon having flexible circuits disposed thereon.

FIG. 6 illustrates one embodiment of a flexible circuit for use on the renal nerve modulation balloon.

FIG. 7 is a Field Emission Scanning Electron Microscopy (FESEM) image illustrating a balloon surface prior to laser etching.

FIG. 8 is a Field Emission Scanning Electron Microscopy (FESEM) image illustrating the surface roughness of the body portion of a 6 mm balloon obtained using laser etching techniques.

FIG. 9 is an FESEM image illustrating the surface roughness of the cones of a 6 mm balloon obtained using laser etching techniques.

FIG. 10 is a FESEM image illustrating the surface roughness of the waist portions of a 6 mm balloon obtained using laser etching techniques.

FIG. 11 is a FESEM image illustrating the surface roughness of a flexible circuit

FIG. 12 is a FESEM image illustrating the surface roughness of a flexible circuit obtained using laser etching techniques.

FIGS. 13A-13I are FESEM images illustrating the nodule height of nine different laser etched balloon samples.

FIG. 14 is a graph illustrating the surface roughness of the same nine different balloon samples as in FIG. 13.

FIG. 15 is a graph illustrating the burst strength of a non-textured balloon versus the burst strength of an optimally textured balloon as determined by the study of the same nine balloon samples as in FIGS. 13A-13I and 14.

FIG. 16 is a graph illustrating a rating based on delamination of samples of polyimide flexible circuits adhered to both non-textured and textured balloons after simulated use.

FIG. 17 is a graph illustrating simulated use testing results of samples of polyimide flexible circuits adhered to both non-textured and textured balloons after simulated use.

FIGS. 18 and 19 are graphs illustrating the effect of adhesive thickness on t-peel values and mode of bond failure.

### DETAILED DESCRIPTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The present disclosure relates to balloon for renal nerve modulation comprising a balloon wall having an interior and an exterior surface and flex circuits adhesively bonded to the exterior of a balloon. Renal nerve modulation or renal denervation is sometimes used to treat conditions relating to hypertension and/or congestive heart failure.

While the devices and methods disclosed herein are discussed relative to renal nerve modulation, it is contemplated that these devices and methods may be employed in other treatments as well.

The devices and methods according to the disclosure involve the delivery of radiofrequency energy to the renal nerve to temporarily or permanently modify nerve function.

In alternative embodiments, other sources of energy such as ultrasound energy, microwave energy or direct heating elements may be employed for renal artery denervation.

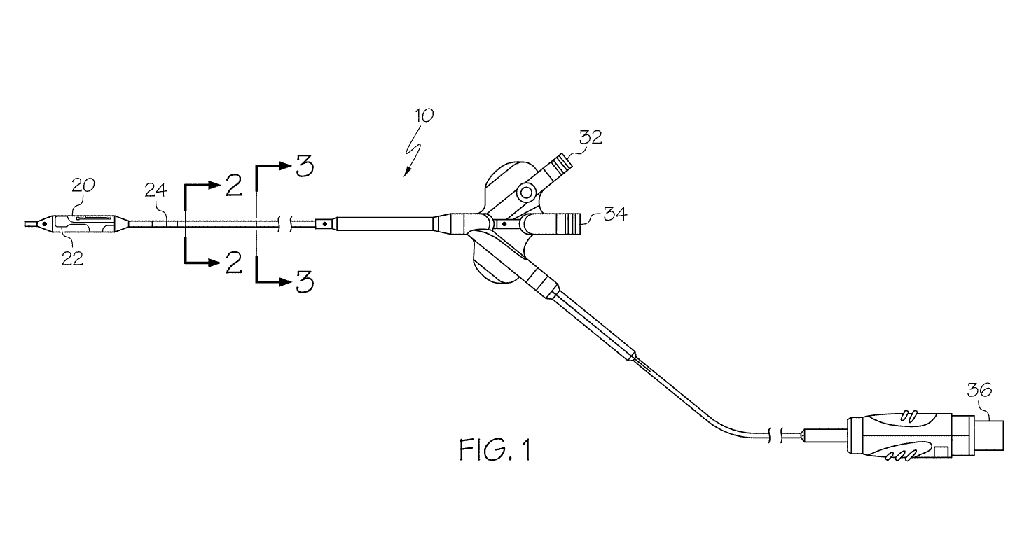

Turning now the drawings, FIG. 1 is a side view of a catheter 10 having a balloon 20 for renal nerve modulation disposed at the distal end thereof. Catheter 10 includes a port 32 for inflation fluid, guidewire lumen 34 and electric plug 36 to a generator.

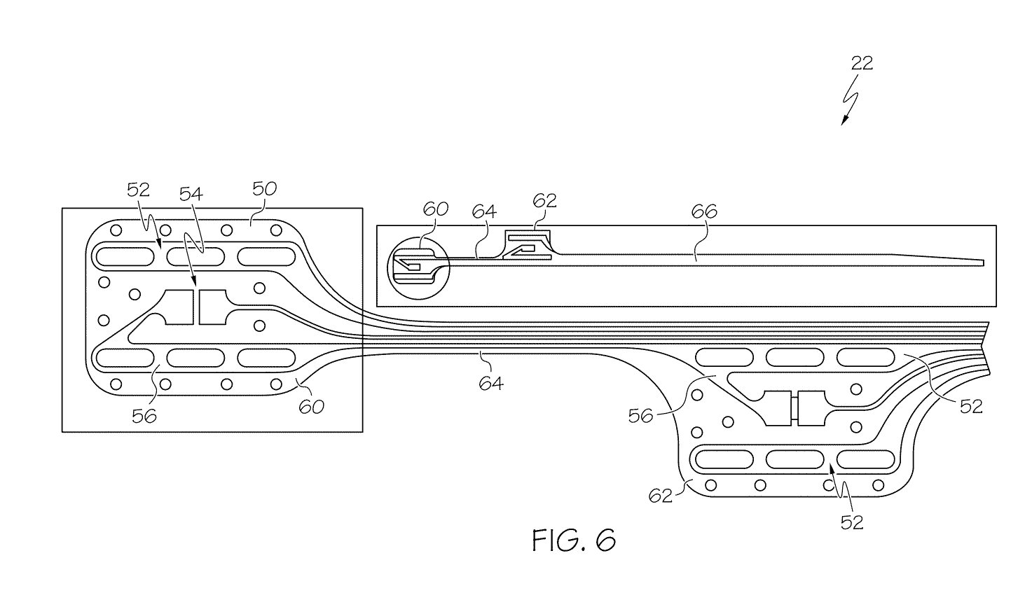

Balloon 20 includes flexible circuits 22 disposed thereon. Balloon 20 is a radially expandable balloon. Balloon 20 is delivered to a treatment site in a patient's vasculature and inflated with fluid supplied through port 32 during use. The balloon is bonded at the distal end to the distal end of an outer shaft 24 and at the proximal end to an inner catheter shaft. Each flexible circuit 22, explained in more detail with respect to FIG. 6 may contain two pairs of electrodes connected to a power supply at the proximal end of the device via wires disposed within or along the outer shaft 24.

FIG. 2 is a cross-sectional view taken at section 2-2 in FIG. 1 illustrating the outer shaft 24 of catheter 10, electrodes 26 disposed within outer shaft 24, inflation lumen 28 and guidewire lumen 30.

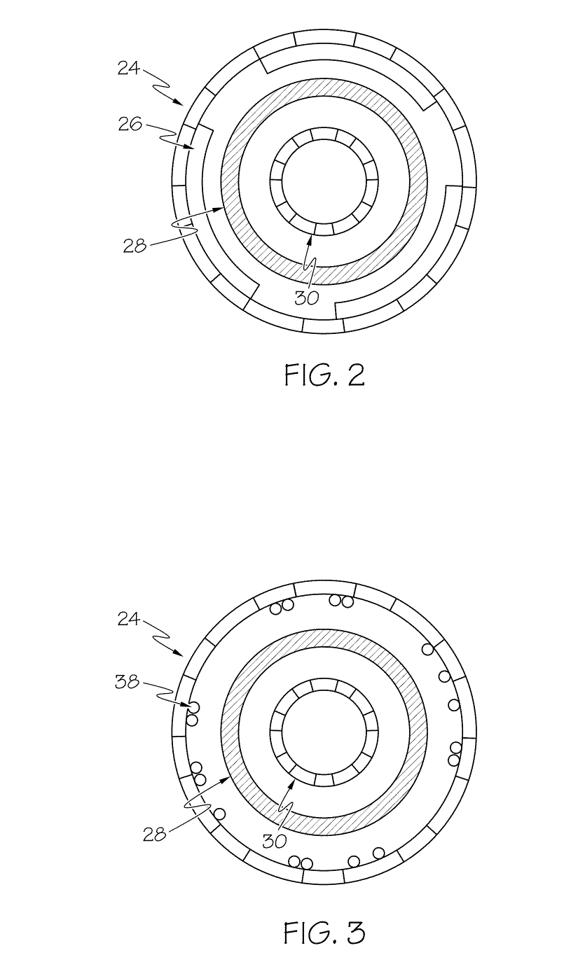

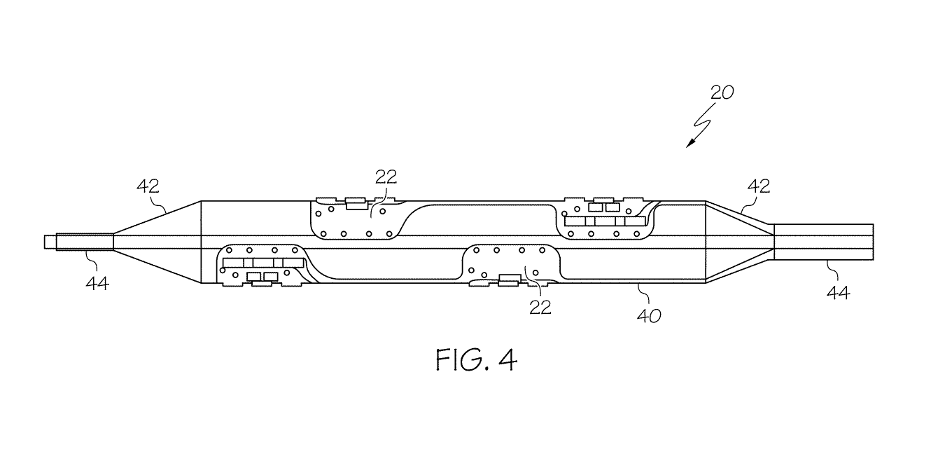

FIG. 3 is a radial cross-sectional view taken at section 3-3 in FIG. 1 further illustrating wires 38 at are distally coupled to electrodes 26. These wires provide power and grounds for the temperature sensors and ablation electrodes. FIG. 4 is a side view of one embodiment of a balloon 20 for renal nerve modulation illustrating two flexible circuits 22 disposed therein. FIG. 4 is illustrative of a balloon that is 4 mm in diameter. Balloon 20 includes a body portion 40, a proximal cone portion 42, a proximal waist portion 44, a distal cone portion 43 and a distal waist portion 45.

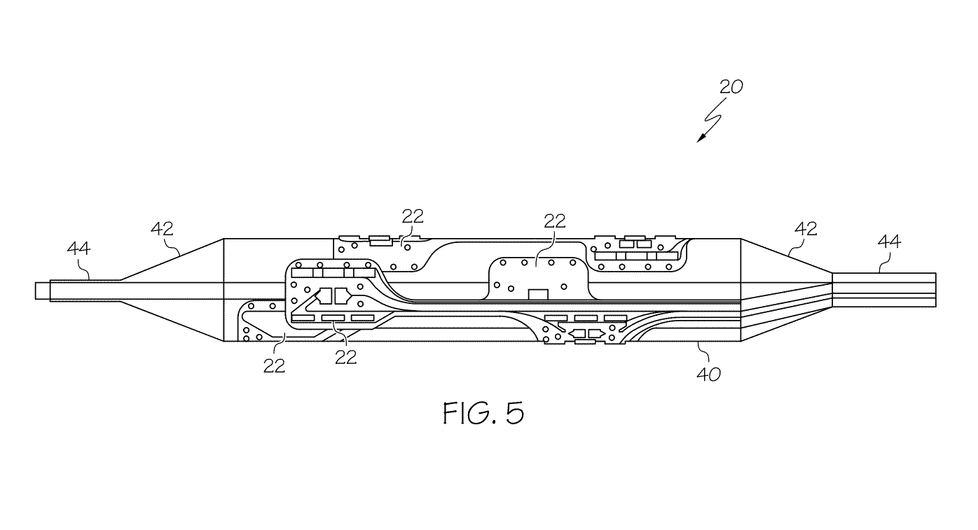

FIG. 5 is a side view of one embodiment of a balloon 20 for renal nerve modulation illustrating four flexible circuits 22 disposed thereon. Alternatively, balloon 20 may have 1, 2, 3, 5, 6 or more flexible circuits disposed thereon.

Larger balloons of 5, 6, 7 or 8 mm diameter, may include a larger number of flexible circuits such as 3 or more flexible circuits.

In this embodiment, each flexible circuit is formed from a polymer base material 50, in this embodiment, Kapton® polyimide available from DuPont™ in Wilmington, Del.

Each flexible circuit 22, as illustrated in FIG. 6, has two electrode pairs 52, coupled to a thermistor 54 mounted on copper traces for individual temperature control feedback, and a shared ground 56. In this embodiment, electrodes 52 are formed from gold but can be formed from any suitable material such as platinum, stainless steel, cobalt alloys, and other non-oxidizing materials. Each flexible circuit forms a complete bipolar circuit which is beneficial because there are reduced treatment variation effects from non-targeted body tissue proximity such as fatty tissue, or energy loss due to variations in blood flow cooling effects.

The flexible circuit 22 further includes copper wires which may be lithographically printed thereon.

In this embodiment, the electrodes are spatially arranged for 360° circumferential coverage and spaced longitudinally with an offset about the balloon resulting in coverage of the artery that avoids ablating a single circumferential ring which might cause injury to healthy renal tissue, potentially resulting in vessel trauma and stenosis. Thus, the discrete treatment locations provided by the individual flexible circuits 22 are spatially arranged to provide full circumferential coverage of the vessel for denervation while minimizing negative impact to the healthy vessel.

The balloon 20 may be formed of noncompliant polymer materials or semi-compliant or compliant polymer materials.

Compliant balloons are made from relatively soft or flexible polymeric materials. Examples of these materials are thermoplastic polymers, thermoplastic elastomers, polyethylene (high density, low density, intermediate density, linear low density), various copolymers and blends of polyethylene, ionomers, polyesters, polyurethanes, polycarbonates, polyamides, polyvinyl chloride or acrylonitrile-butadiene-styrene copolymers. A suitable copolymer material, polyolefin material is available from E. I. DuPont de Nemours and Co. (Wilmington, Del.), under the trade name Surlyn® Ionomer.

Intermediate compliant balloons are made of polyether-block-amide (PEBA) copolymers and nylon materials.

Non-compliant balloons are made from relatively rigid or stiff polymeric materials. These materials are thermoplastic polymers and thermoset polymeric materials. Some examples of such materials are poly(ethylene terephthalate), polyimide, thermoplastic polyimide, polyamides, polyesters, polycarbonates, polyphenylene sulfides, polypropylene and rigid polyurethanes. Non-Compliant balloons made from poly(ethylene terephthalate) are commonly referred to as PET balloons.

In some embodiments, the balloon 20 is formed of a non-compliant polymer material such as polyethylene terephthalate (PET).

The flexible circuit 22 is disposed on the outer surface of the balloon 20. Suitably, the flexible circuit 22 is disposed on the balloon 20 outer surface via an adhesive bond.

A suitable adhesive may be employed providing it is a biocompatible medical grade adhesive including thermoplastic and thermoset adhesives.

In some embodiments, the adhesive is a thermoset adhesive.

In some embodiments, the adhesive is an ultraviolet (UV) curable adhesive.

In one embodiment, the adhesive is a urethane-acrylic adhesive.

One example of a commercially available medical grade urethane-acrylic adhesive is Dymax® 204 CTH available from Dymax® Corporation in Torrington, Conn.

The adhesive may be applied to the balloon 20, the flexible circuit 22, or both. Suitably, the adhesive is disposed at least on the portion of the balloon and/or flexible circuit which are in contact with one another.

It has been found that electrode attachment robustness can be improved by texturing the outer surface of the balloon 20 and/or the inner surface or bonding surface of the flexible circuit 22.

In some embodiments a laser is employed to etch the surface of the balloon 20 and/or flexible circuit 22 which results in laser induced microstructures of on the surface of the balloon 20 and/or flexible circuit 22 to improve adhesive surface wetting, mechanical anchorage of the adhesive, and ultimately the adhesion of the flexible circuit 22 to the balloon 20.

In some embodiments, the surface of the balloon 20 is textured on the portion upon which the flexible circuit 22 is disposed, and in some embodiments, the balloon 20 is textured to extend just beyond the periphery of the flexible circuit 22. The periphery is defined by the outline or outer edge of the flexible circuit 22.

In particular embodiments, an excimer laser is employed to texturize or roughen the balloon surface to which the polyimide flexible circuit 22 is adhered.

It has been found that optimal average surface roughness is about 0.25 μm to about 0.45 μm. The average surface roughness as used herein is determined by the following equation:

$${R}_{avg} = {\sum }_{n = 1}^{N} \frac{\vert {Z}_{n} - \overline{Z} \vert}{N}$$

Where Z̅ is the surface height relative to the center plane which is determined by equating the volumes enclosed by the image of the surface above and below the plane and N is the number of points in the sample area.

Surface roughness of 0.20 μm or less fails to provide adequate adhesion between the flexible circuit 22 and the balloon 20 while surface roughness of 0.45 μm or higher may result in a weakening of the balloon wall with a decrease burst strength. Burst strength is calculated as follows:

Burst Strength=P×D/2t

where P=internal pressure when the balloon bursts (kg/cm²) (psi); D is the exterior diameter (mm) of the balloon 20 when a pressure of 6.2 kg/cm² (88 psi) is applied; and t is the wall thickness of the portion of the balloon 20 with the larger exterior diameter.

Suitably, the height of the laser induced microstructures or nodules is about 1 μm to about 4 μm.

In achieving the desired surface roughness, about 10-20% of the polymer material forming the balloon wall is at least somewhat removed, leaving discrete surface features or nodules. Removing more than this amount of material can negatively impact the burst strength of the balloon 20. For example, a surface roughness of about 0.3 μm results in a balloon having 10-20% of the polymer material forming the balloon wall removed. This is for a balloon having a 1× wall thickness of about 0.0005″ or about 13 μm and a resultant nodule height of about 1 μm to about 3 μm.

Surface roughness and nodule height is approximated using Field Emission Scanning Electron Microscopy (FESEM). FESEM employs high magnification of up to 50,000×, with about 1-15,000× being desirable.

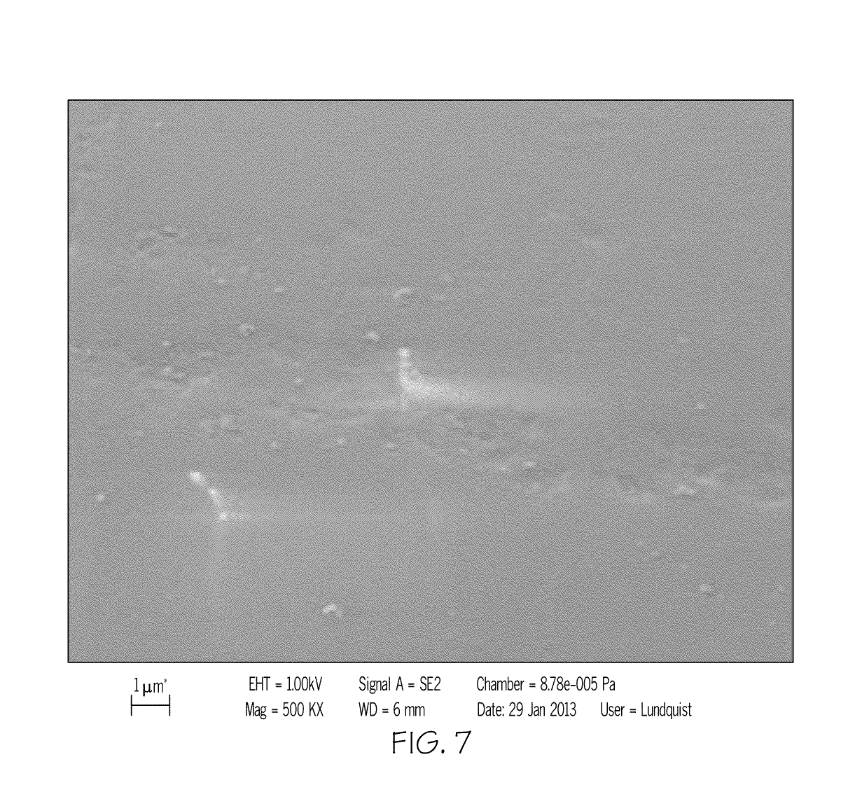

For comparative purposes only, FIG. 7 is a FESEM image illustrating a balloon surface prior to laser etching.

FIG. 8 is a FESEM image illustrating the surface of a 6 mm polyethylene terephthalate balloon with single wall thickness of 0.0005″. FIG. 8 illustrates the balloon body after laser etching with an excimer laser. The laser induced microstructures or nodules are clearly visible on the surface of the balloon 20.

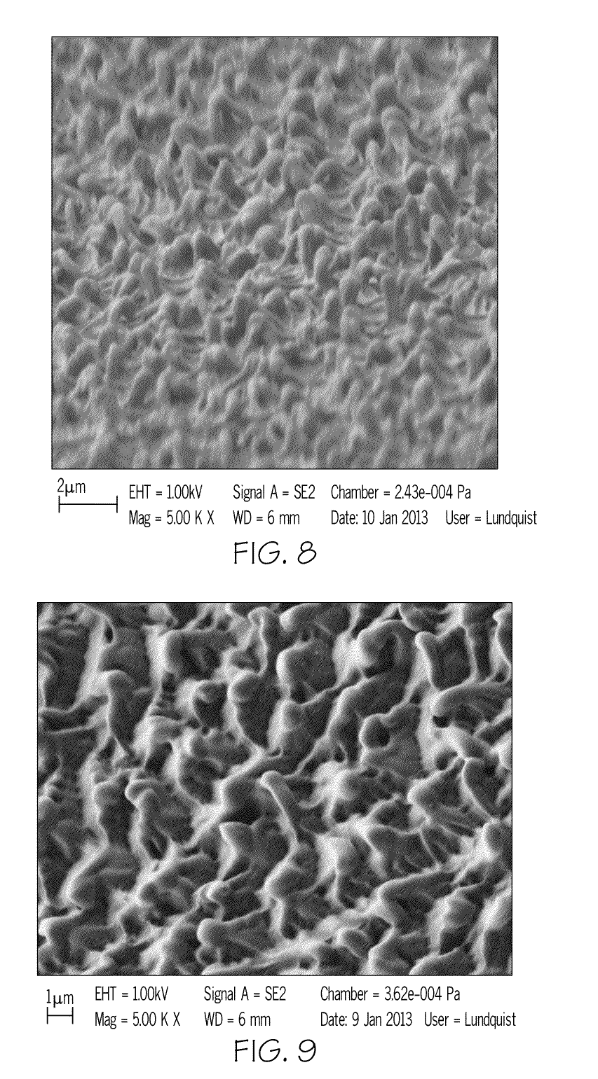

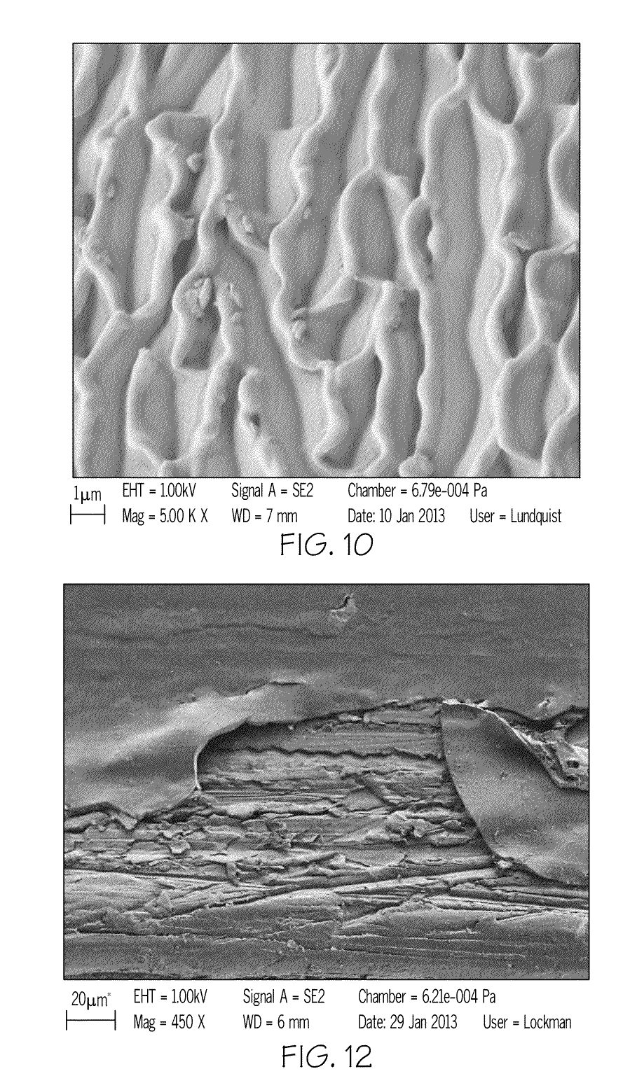

FIGS. 9 and 10 are FESEM images of a 6 mm polyethylene terephthalate balloon with a single wall thickness of 0.0005″. The cone and waist portions are shown in FIGS. 8 and 9 respectively after laser etching with an excimer laser. The laser etching achieved on the cone and waist portions is characteristically different from that of the balloon body. It is surmised that the difference is a result of different orientation of the PET in the cones and waist. The body of the balloon 20 is more biaxially oriented whereas the cones and waists ore more longitudinally oriented.

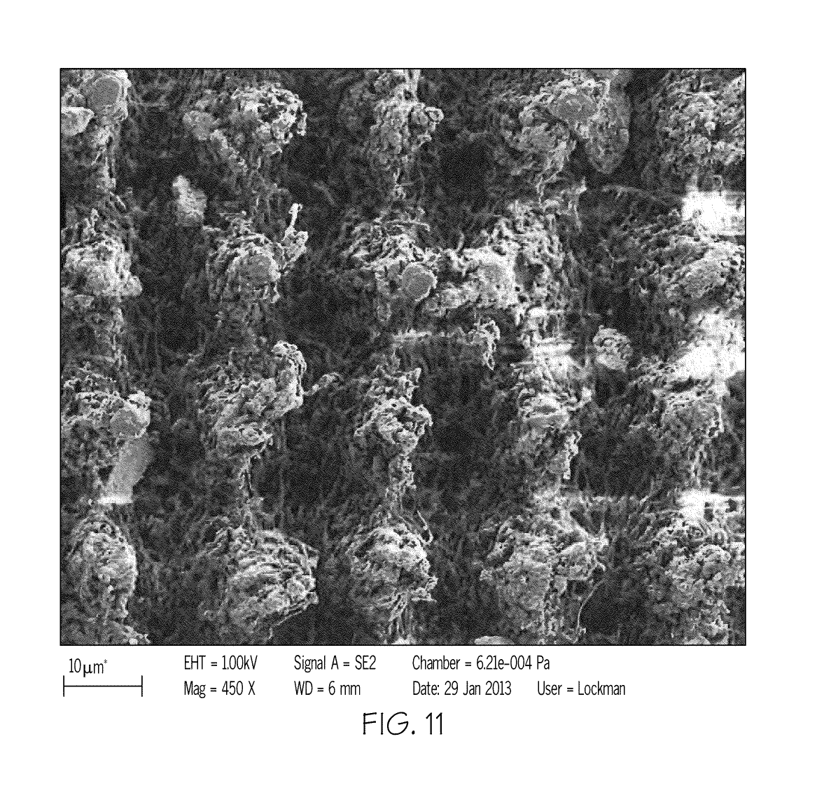

Alternatively or in combination, the inner surface of the flexible circuit may be laser etched in the same manner to achieve surface roughness. FIG. 11 is a FESEM image of a flexible circuit with a polyimide base after laser etching. Surface roughness or nodules are clearly visible from the FESEM image.

FIG. 12 is a FESEM image illustrating a prior art method of hand sanding the flexible circuit having a polyimide base which results in scratches and irregular surface morphology as well as inferior surface roughness.

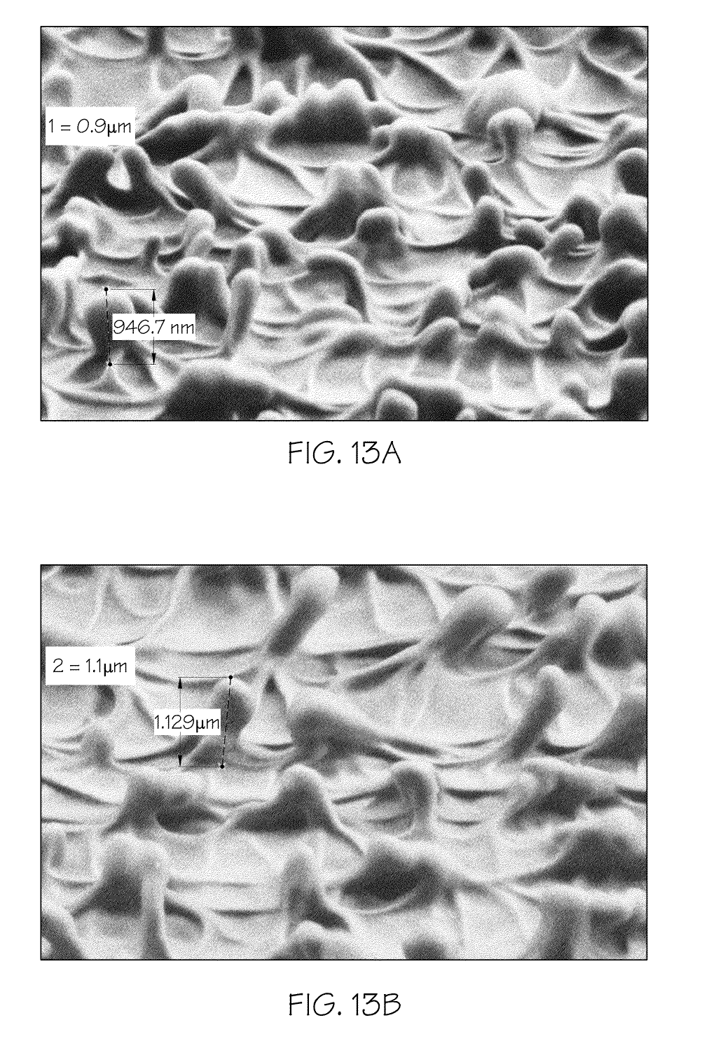

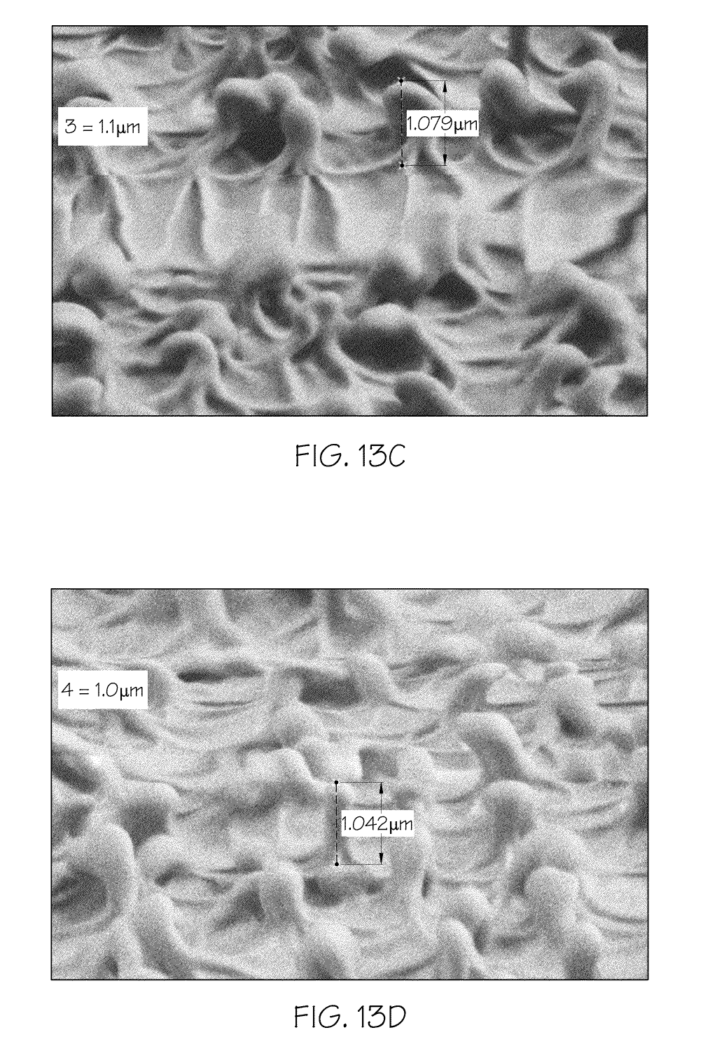

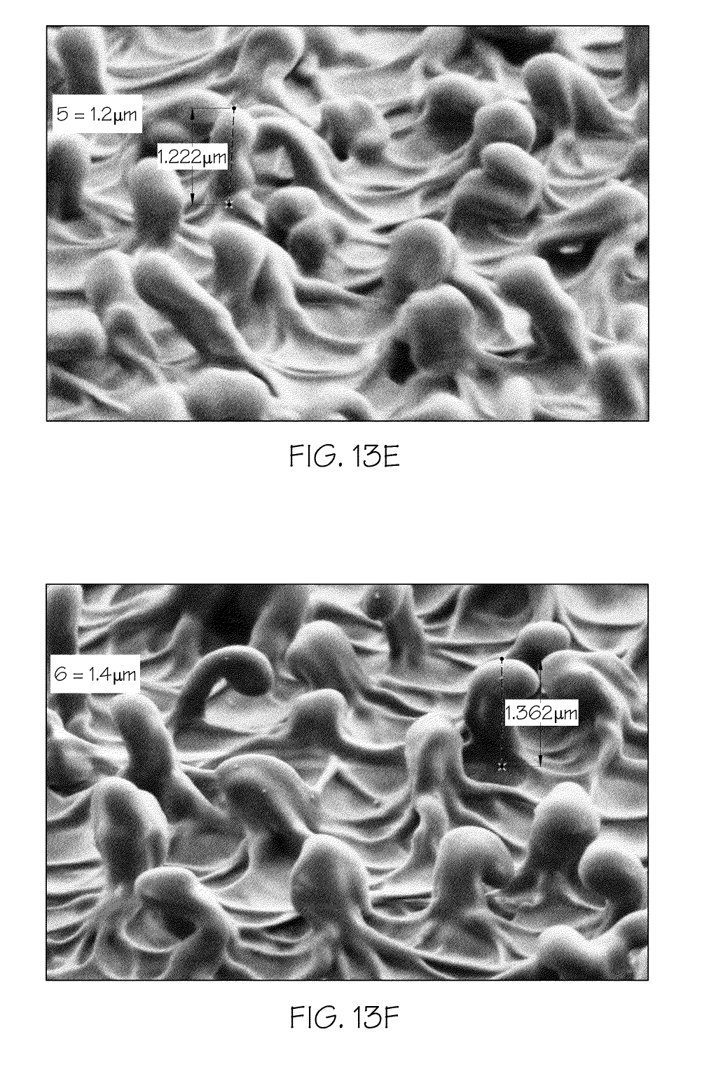

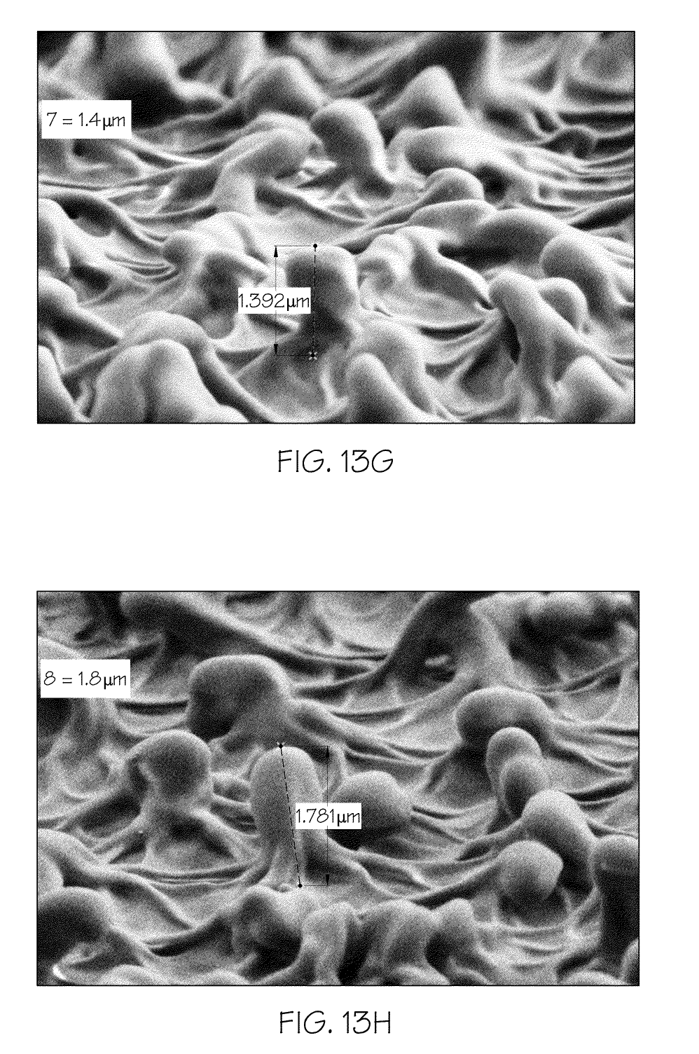

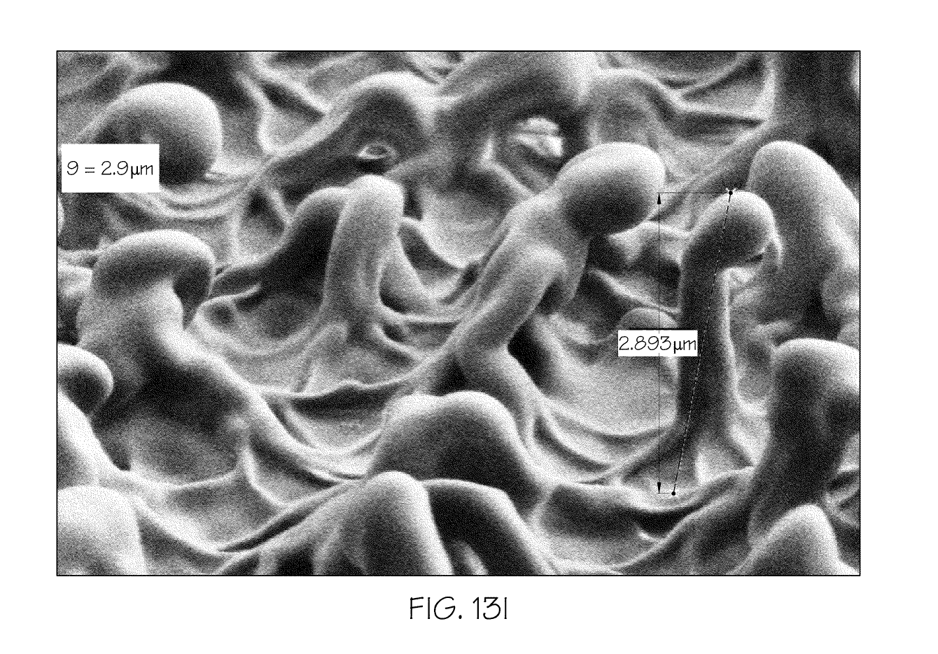

FIGS. 13A-13I are a FESEM images illustrating nine different sample studies of a 6 mm polyethylene terephthalate balloon body having a single wall thickness of 0.0005″ illustrating laser induced microstructures formed with increasing energy and increasing overlapping or number of passes with the excimer laser with FIG. 13I having the maximum overlapping and energy employed in this experiment. As can be seen from FIGS. 13A-13D, for example, using a lower energy level and less overlapping, produces nodules having a height of about 1 μm. Nodule heights of about 3 μm were achieved with increasing energy and rotational overlap as shown by the sample in FIG. 13I.

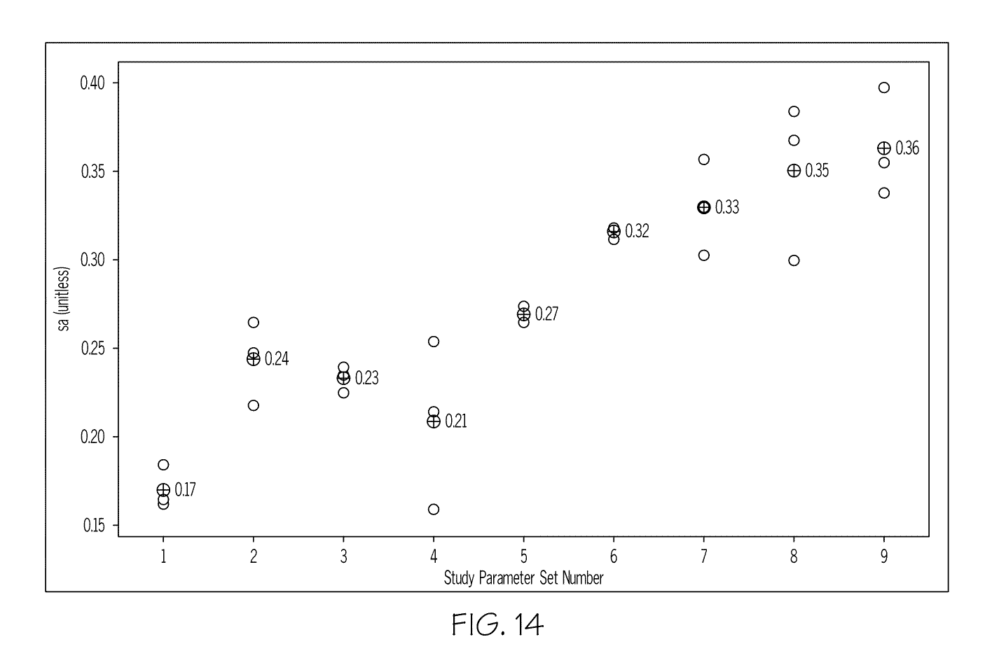

FIG. 14 is a graph illustrating that optimum surface roughness in the desired range is achieved using nominal energy/nominal rotational overlap and high energy/high rotational overlap, while the surface roughness achieved with low energy and low rotational overlap failed to achieve the desired surface roughness.

The balloons were laser etched at 194 nm UV wavelength output with argon noble gas plus fluoride halide active gas pulse discharge excimer laser process. Key process parameters are rotational overlap and applied energy. Nominal energy and nominal rotational overlap and high energy and high rotational overlap achieve surface roughness values in the desired range of 0.25 μm to 0.45 μm while low energy and low rotational overlap failed to achieve the desired results.

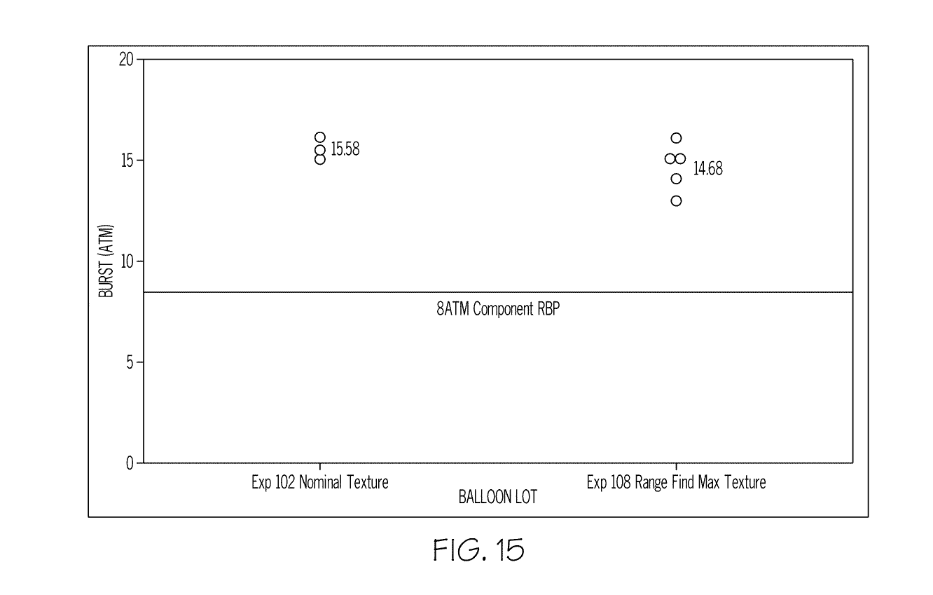

FIG. 15 is a graph showing balloon burst in ATM of a non-textured balloon and the balloon burst of a textured balloon having a surface roughness of about 0.30 μm, the surface of which is illustrated in FIG. 13I. Texturing within the desired range does not negatively impact balloon burst strength.

Optionally, a protective bead of adhesive is applied around the perimeter of the flexible circuit after the flexible circuit has been adhered to the balloon. The present disclosure can also be employed to improve the adhesion of the adhesive bead by laser texturing around the perimeter of the balloon outer surface around the flexible circuit and/or the top side of the flex circuit around the perimeter of the flexible circuit.

The catheter shaft to which the waist portion of the balloon is adhered may also be textured according to the method of the present application. A catheter shaft may also be bonded using a UV curable adhesive. Suitably, when texturing a catheter shaft bonded in this fashion, suitably the texturing is conducted in circumferential bands, with non-textured bands in between, to allow for UV transmission throughout the bond length.

### EXAMPLES

### T-Peel Test Method

The samples were prepared for t-peel testing by affixing the two substrates to glass slides. The cones of the balloon were cut off and the balloon was slit open into a flat rectangle. The polyimide substrate used for making the flexible circuits was cut into a rectangle. Care was taken to avoid contamination of the surfaces to be adhered together.

Approximately 10 mm of the substrate extend from the edge of the glass slide leaving enough material for the tensile tester grips. A 0.005″ tape was affixed to both sides of the polyimide rectangle to account for the thickness of the balloon and polyimide substrates, leaving a gap of 0.002″ for the adhesive. Adhesive was applied to the polyimide, the slide with the balloon was placed on top such that the balloon is aligned with the polyimide rectangle, and slight pressure is employed to spread the adhesive evenly. The adhesive employed was Dymax® 204 CTH urethane-acrylic adhesive available from Dymax® Corporation in Torrington, Conn. Excess adhesive is wicked away using a paper sheet, again making sure the two substrates were not in intimate contact. Ultraviolet radiation was delivered through the glass slide and the balloon, taking into account any attenuation. Estimated dose was 2 W/cm² for 60 seconds using an Omnicure LX400 LED source, metered at 365 nm wavelength. Lower dosages of 200 mW/cm² for 3 seconds has been found to be sufficient for adhesive cure.

After cure, the samples were allowed to sit at ambient temperature for at least 10 hours. The rectangular sample was cut into strips with a twin-blade cutter, leaving out both of the side to avoid any edge effect. The tensile testing was carried out at a crosshead speed of 200 mm/minute.

### Withdrawal Force Test Method

Polyimide flexible circuits are bonded to catheter balloons and cured as described above, except that the balloons are left intact. The balloon catheters are then prepped by attaching a syringe loaded with saline to the inflation port of the catheter. A vacuum was pulled with the syringe while the catheter is hanging distal end down to remove any air from the catheter system while filing the inflation lumen with an incompressible liquid. The balloons catheters were then inserted through a guide with specified fixturing. In this example, the guide inner diameter (ID) size was a 7F guide sheath having a minimum ID of 2.33 mm. The size of the guide sheath can be changed as desired. The balloons were inflated to rated burst pressure. In this example, the rated burst pressure was 5 ATM with a nominal pressure of 3 ATM. The rated burst pressure of renal denervation balloons varies. The balloon were then placed into the test media and run electrically using a withdrawal speed of 50 cm/minute. The balloon was deflated and the flexible circuit evaluated and the withdrawal force calculated. The balloon is inserted and withdrawn from the catheter 7 times.

### Surface Characterization Using Optical Interferometry (VSI)

A white light interferometer was used, specifically, a Veeco NT9800 Optical Profiler. All balloons characterized by VSI were studied at magnifications of 50× and 100× (the equipment maximum). Like AFM, values for surface roughness and peak (nodule) height and surface area were captured for the textured balloon surfaces.

### Example 1

This example illustrates the impact of texturing on the adhesion of the flexible circuit having a polyimide base to a polyethylene terephthalate balloon. Delamination of the flexible circuit from the balloon is determined. The testing involved simulated use of the devices. The steps include insertion of a balloon into a sheath, activation of the device (electrically), and withdrawal of the balloon from the sheath. The insertion/activation/withdrawal cycle is repeated seven times. The balloons are visually examined and given a score of 0-2 with 0 being no delamination, 1 being partial delamination and 2 being total delamination of the flexible circuit from the balloon.

A tensile testing machine with a load cell is used to measure insertion and withdrawal forces.

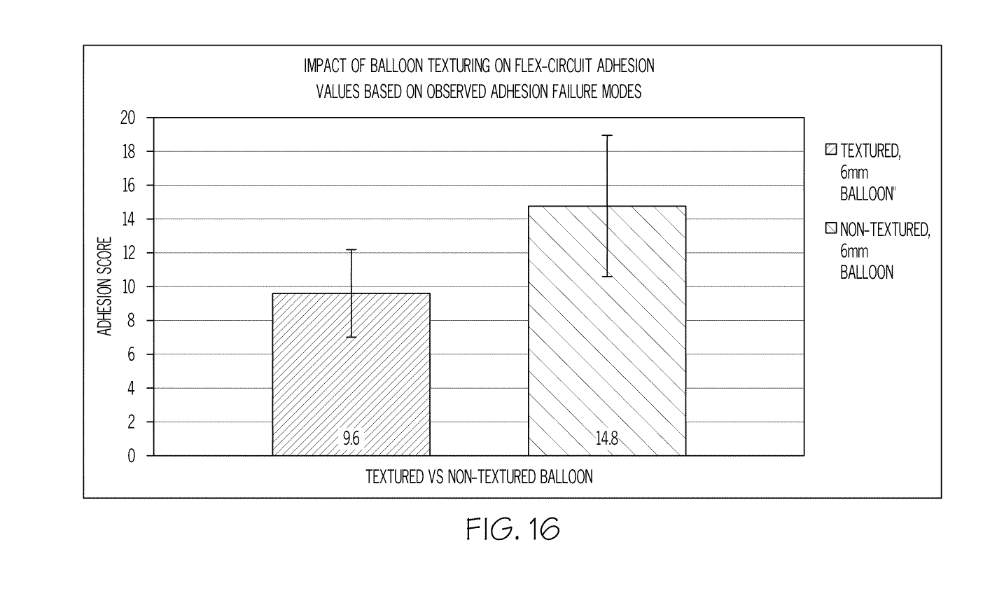

The results are shown in FIG. 16. As can be seen from the graph, balloons having a textured surface according to the disclosure exhibited much less delamination after 7 cycles of insertion and withdrawal of the guide sheath.

As can be seen from the graph, texturing reduces lifting of the flexible circuits from the balloon upon simulation use.

Non-textured balloons accounted for the majority of the severity scores (poor adhesion) above 2 and all scores above 3.

### Example 2

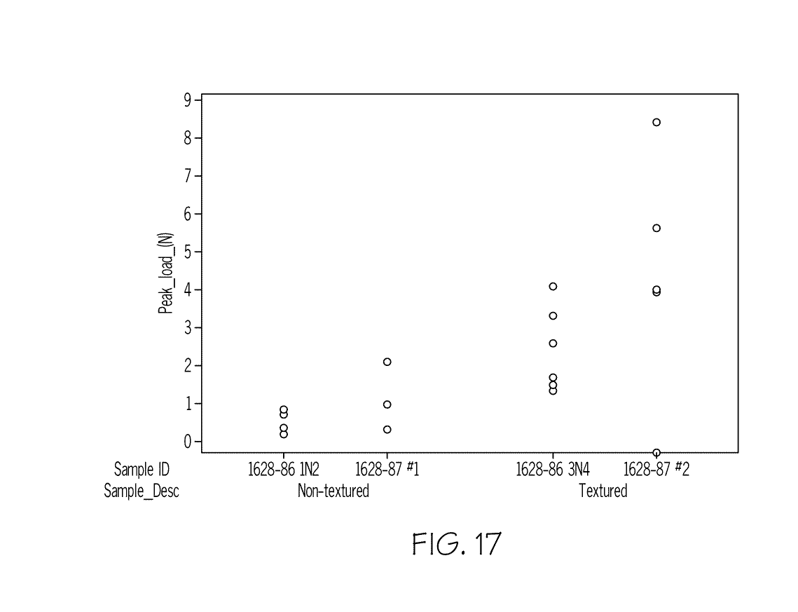

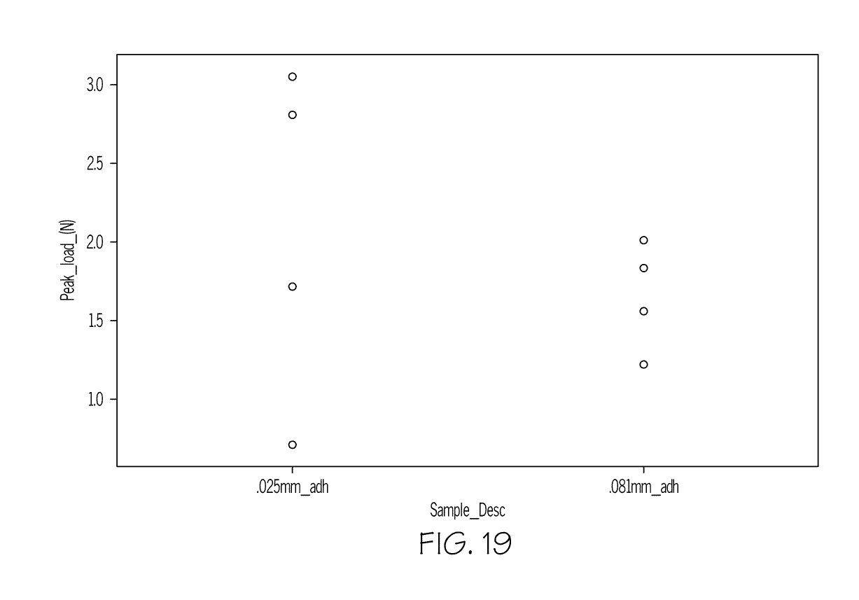

This example illustrates peel force testing between a flexible circuit having a polyimide base and non-textured versus textured polyethylene terephthalate balloons having a single wall thickness of 0.0005″. The results are illustrated in FIGS. 17-19. Texturing results in a much higher t-peel value than a non-textured balloon as shown in FIG. 17.

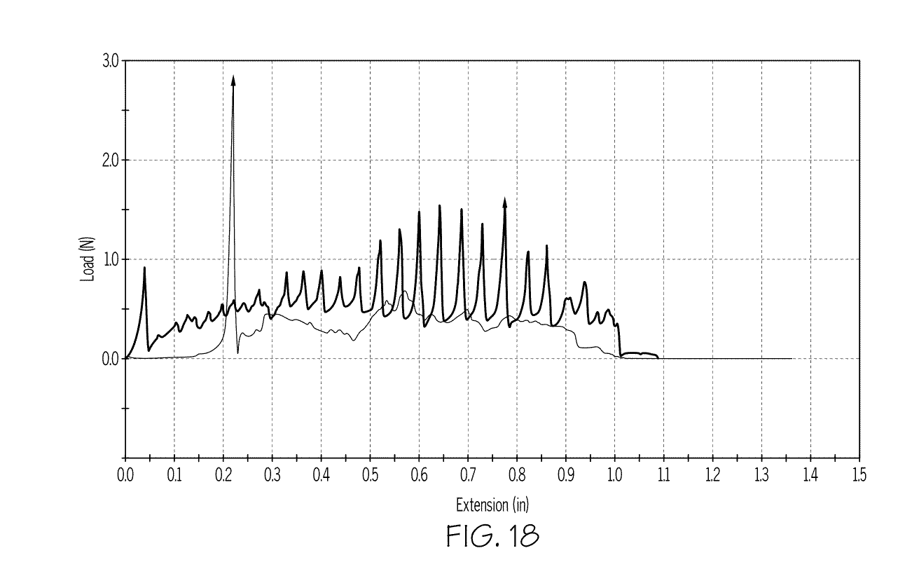

It was further determined that using either a thick or a thin layer of adhesive resulted in a similar maximum t-peel force but the failure mechanisms were different. A thick layer of adhesive (e.g., about 3 mils) exhibited a “zipper like” or cohesive failure mode whereas a thin layer of adhesive (e.g., about 1 mil) exhibited a high initial peak as can be seen from FIGS. 17 and 18.

The present disclosure improves surface roughness over previous methods and achieves improved bonding of the flexible circuit to the balloon outer surface.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All published documents, including all US patent documents and US patent publications, mentioned anywhere in this application are hereby expressly incorporated herein by reference in their entirety. Any copending patent applications, mentioned anywhere in this application are also hereby expressly incorporated herein by reference in their entirety. Citation or discussion of a reference herein shall not be construed as an admission that such is prior art.

U.S. Patent Application Pub No. US 2014/0128859A1 is herein incorporated by reference.

What is claimed is:

1. A balloon for renal nerve modulation, the balloon comprising:
a polymer material forming a balloon wall having an interior surface and an exterior surface; and
one or more flexible circuits adhesively bonded to the exterior surface of the balloon wall;
wherein the exterior surface comprises laser induced microstructures on at least a portion of said exterior surface; and
wherein said laser induced microstructures have an average surface roughness of about 0.25 μm to about 0.45 μm.

2. The balloon of claim 1, wherein said laser induced microstructures have a height of about 1 μm to about 3 μm.

3. The balloon of claim 1, wherein said flexible circuits are adhesively bonded to the exterior surface of the balloon wall with a thermoset adhesive.

4. The balloon of claim 3, wherein said thermoset adhesive is a UV cured adhesive.

5. The balloon of claim 4, wherein said adhesive is a urethane-acrylic adhesive.

6. The balloon of claim 1, wherein said polymer material comprises a non-compliant polymer material.

7. The balloon of claim 6, wherein said non-compliant polymer material is polyethylene terephthalate.

8. The balloon of claim 1, wherein said polymer material comprises a semi-compliant or a compliant polymer material.

9. The balloon of claim 1, where said flexible circuits comprise a polyimide material having copper lithographically printed thereon.

10. The balloon of claim 1, wherein said flexible circuits comprise laser induced microstructures.

11. The balloon of claim 1, wherein said balloon comprises a body, a proximal waist, a proximal cone, a distal waist and a distal cone and said at least a portion of said surface comprises said body, said proximal waist and said proximal cone.

12. The balloon of claim 1, wherein said flexible circuits are defined by a periphery, said at least a portion of said exterior surface comprises the surface underneath of said flexible circuits and extending just beyond the periphery of the flexible circuits.

13. The balloon of claim 1, wherein said balloon has a diameter of about 4 mm to about 8 mm.

14. The balloon of claim 1, comprising 2 to 4 flexible circuits.

15. A flexible circuit, the flexible circuit having an inner surface and an outer surface, the inner surface of the flexible circuit is adhesively bonded to at least a portion of an outer surface of a balloon for renal nerve modulation, the flexible circuit comprising laser induced microstructures on said inner surface that is adhesively bonded to said at least a portion of the outer surface of the balloon.

16. The flexible circuit of claim 15, wherein the outer surface of the balloon comprises laser induced microstructures.

17. A method of making a balloon for renal nerve modulation, the method comprising:
providing a balloon having a balloon wall, the balloon wall comprising a polymer material, the balloon having a body, waist and cone portions, the balloon having an interior surface and an exterior surface;
removing material from the exterior surface of the balloon by laser etching, the laser etching forms laser induced microstructures in the exterior surface of the balloon;
wherein said laser induced microstructures have an average surface roughness of about 0.25 μm to about 0.45 μm;
adhesively bonding flexible circuits to the exterior surface of the balloon on at least a portion of the balloon comprising said laser induced microstructures.

18. The method of claim 17, wherein said removing step comprises removing about 10% of the polymer material.

19. The method of claim 17, wherein said laser etching comprises excimer laser etching.

20. The method of claim 17, wherein said laser induced microstructures have a base width of about 0.25 μm to about 0.45 μm and a height of about 1 μm to about 3 μm.
